(19) United States
(12) Patent Application Publication
(10) Pub. No.: US 2022/0175072 A1
(43) Pub. Date: Jun. 9, 2022

(54) Safety Headwear with Electronic Monitoring

(71) Applicant: Milwaukee Electric Tool Corporation, Brookfield, WI (US)

(72) Inventors: Benjamin T. Jones, St. Francis, WI (US); Steven B. Sedig, Fox Point, WI (US)

(21) Appl. No.: 17/554,854

(22) Filed: Dec. 17, 2021

Related U.S. Application Data
(63) Continuation of application No. PCT/US2021/061588, filed on Dec. 2, 2021.
(60) Provisional application No. 63/122,301, filed on Dec. 7, 2020.

(51) Int. Cl. A42B 3/04; A42B 3/28

(52) U.S. Cl. CPC A42B 3/0433; A42B 3/288

(57) ABSTRACT

Various embodiments of safety headwear are provided. The safety headwear includes one or more of a hard hat, such as a safety helmet, and a respirator. The safety headwear monitors one or more conditions, such as biometric and/or atmospheric conditions. The safety headwear analyzes the results of the monitoring to determine whether to generate an alarm to the wearer of the safety headwear.

### CROSS-REFERENCE TO RELATED PATENT APPLICATIONS

The present application is a continuation of International Application No. PCT/US2021/061588, filed Dec. 2, 2021, which claims the benefit of and priority to U.S. Provisional Application No. 63/122,301, filed on Dec. 7, 2020, which are incorporated herein by reference in their entireties.

### BACKGROUND OF THE INVENTION

The present invention relates generally to the field of safety equipment. The present invention relates specifically to a hard hat and/or respirator with an electronic monitoring system. Hard hats are often used to protect the wearer, and respirators are often used to protect a user from breathing particles and dust in the air.

### SUMMARY OF THE INVENTION

One embodiment of the invention relates to a hard hat including an outer shell, an outer surface of the outer shell, an inner surface of the outer shell, a biometric measuring device, and an environmental measuring device. The inner surface defines a cavity configured to receive a head of a person wearing the hard hat. The biometric measuring device is supported by the outer shell and is configured to measure a biometric characteristic of the person wearing the hard hat. The environmental measuring device is supported by the outer shell and is configured to measure an atmospheric condition.

Another embodiment of the invention relates to a respirator including a body, a gasket, a filter, an air pressure measuring device, and a monitoring unit. The gasket is coupled to the body and is configured to engage against a face of a person wearing the respirator to define a secured area between the respirator and the face of the person. The filter is coupled to the body and is configured to remove particulates from air passing through the filter to the secured area. The air pressure measuring device is supported by the body and configured to measure an air pressure measurement within the secured area and generate a signal indicating the measured air pressure measurement. The monitoring unit is supported by the body and configured to receive the signal from the air pressure measuring device indicating the air pressure measurement. The monitoring unit is configured to generate an alarm in response to detecting an error condition based on analyzing the air pressure measurement.

Another embodiment of the invention relates to a hard hat system including a respirator and a hard hat. The respirator includes a body, a gasket, a filter, and an air pressure measuring device. The gasket is coupled to the body and configured to engage against a face of a person wearing the respirator to define a secured area between the respirator and the face of the person. The filter is coupled to the body and configured to remove particulates from air passing through the filter to the secured area. The air pressure measuring device is supported by the body and configured to generate a first signal indicating an air pressure measurement. The hard hat includes an outer shell, an inner surface of the outer shell, a biometric measuring device supported by the outer shell, and a monitoring unit supported by the outer shell and communicably coupled to both the air pressure measuring device and the biometric measuring device. The inner surface defines a cavity configured to receive a head of the person wearing the hard hat and the respirator. The biometric measuring device is configured to generate a second signal indicating a measurement of a biometric characteristic of the person wearing the hard hat. The monitoring unit is configured to receive and analyze the first signal and the second signal. The monitoring unit is configured to generate a first alarm in response to analyzing one or more of the first signal and the second signal.

Another embodiment of the invention relates to a hard hat including an outer shell, an outer surface of the outer shell, an inner surface of the outer shell, the inner surface defining a cavity configured to receive a head of a person wearing the hard hat, and a monitoring device coupled to the outer shell. The monitoring device measures one or more biometric characteristics of the person wearing the hard hat.

In a specific embodiment, the hard hat includes a processing unit that analyzes the one or more biometric characteristics to generate a risk assessment. The processing unit generates an alarm to the user as a result of analyzing the biometric characteristics. In a specific embodiment, the hard hat includes an environmental monitoring device that measures an atmospheric condition. In a specific embodiment, the atmospheric condition includes one or more of a temperature of ambient air around the hard hat and a humidity of ambient air around the hard hat. In a specific embodiment, the hard hat includes an acceleration monitoring device that measures an acceleration of the hard hat. In a specific embodiment, the one or more biometric characteristics includes a galvanic skin response measurement. In a specific embodiment, the one or more biometric characteristics includes a heart rate of the person wearing the hard hat, a temperature of the person wearing the hard hat, and/or an oxygen saturation (SpO2) of the person wearing the hard hat.

An exemplary method of using an embodiment of the invention includes receiving a signal indicating an air pressure measurement in a secured area between a respirator and a face of a person wearing the respirator, the respirator including a filter configured to remove particulates from air passing through the filter to enter the secured area, analyzing the signal to determine if an error condition is detected, and in response to detecting the error condition, generating an alarm signal.

In a specific embodiment, analyzing the signal includes comparing the air pressure measurement to a predetermined value. The alarm signal is generated as a result of the comparison determining the air pressure measurement is lower than the predetermined value. In a specific embodiment, the method includes receiving a second signal indicating a volume of air transiting the filter. In a specific embodiment, the method includes receiving a signal that uniquely identifies the filter, and generating an alarm to replace the filter as a result of analyzing the air pressure measurement and the volume of air transiting the filter. In a specific embodiment, the second signal is based at least in part on a velocity of air transiting a measurement device in fluid communication with the filter, and a surface area of a projection that deflects in response to air moving past the projection. The measurement device measures an amount of deflection of the projection to determine an estimated velocity of the air transiting the measurement device. In a specific embodiment, the measurement device includes a photodetector that measures the amount of deflection of the projection. In a specific embodiment, the method includes receiving a signal that uniquely identifies the filter, measuring an amount of time the filter has been in use, and generating an alarm signal as a result of the amount of time exceeding a predetermined threshold.

Additional features and advantages will be set forth in the detailed description which follows, and, in part, will be readily apparent to those skilled in the art from the description or recognized by practicing the embodiments as described in the written description included, as well as the appended drawings. It is to be understood that both the foregoing general description and the following detailed description are exemplary.

The accompanying drawings are included to provide further understanding and are incorporated in and constitute a part of this specification. The drawings illustrate one or more embodiments and, together with the description, serve to explain principles and operation of the various embodiments.

### BRIEF DESCRIPTION OF THE DRAWINGS

This application will become more fully understood from the following detailed description, taken in conjunction with the accompanying figures, wherein like reference numerals refer to like elements in which:

FIG. 1 is a perspective view of a hard hat, according to an exemplary embodiment.

FIG. 2 is a detailed side view of a portion of the hard hat of FIG. 1, according to an exemplary embodiment.

FIG. 3 is an exemplary method of operating an electronic monitoring system of the hard hat of FIG. 1, according to an exemplary embodiment.

FIG. 4 is an exemplary chart of measurement results obtained via the electronic monitoring system of the hard hat of FIG. 1, according to an exemplary embodiment.

FIG. 5 is a perspective view of a respirator, according to an exemplary embodiment.

FIG. 6 is a schematic top view of a portion of the respirator of FIG. 5, according to an exemplary embodiment.

FIG. 7 is a schematic side view of a portion of the respirator of FIG. 5, according to an exemplary embodiment.

FIG. 8 is a perspective view of a portion of the respirator of FIG. 5, according to an exemplary embodiment.

FIG. 9 is an exemplary graph of measurement results obtained via an electronic monitoring system of the respirator of FIG. 5, according to an exemplary embodiment.

FIG. 10 is an exemplary graph of measurement results obtained via an electronic monitoring system of the respirator of FIG. 5, according to an exemplary embodiment.

### DETAILED DESCRIPTION

Referring generally to the figures, various embodiments of safety headwear, such as a hard hat and/or a respirator, are shown. Safety headwear provides a layer of protection for the wearer. Described herein are methods to actively monitor the user and the safety headwear to provide the ability to proactively generate signals to improve the performance of the safety headwear. In one example, the safety headwear includes equipment/systems to monitor aspects of the wearer (e.g., heart rate, temperature, SpO2), aspect of the environment (e.g, temperature, humidity) and/or aspects of the safety headwear (e.g., an accelerometer to detect sudden accelerations). Based on the results of the data being monitored, an alarm can be generated for the wearer (e.g., if the heart rate is above a threshold rate for a threshold period of time, generate an alarm for the wearer to rest).

In another example, the safety head gear includes a respirator that monitors performance of the respirator. For example, the respirator monitors variations in pressure while the wearer is breathing to determine whether the respirator is maintaining an airtight seal against the face of the wearer. In another example, the respirator monitors variations in the volume of air passing through the filter and air pressure variations to determine if the filter is clogged and should be replaced.

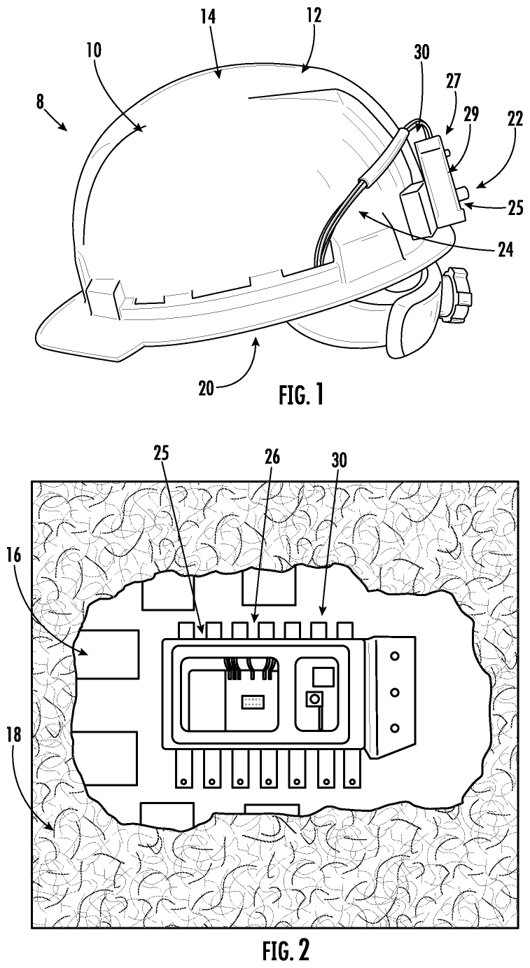

Referring to FIGS. 1-2, safety headwear and/or a safety helmet, shown as hard hat 10, is shown according to an exemplary embodiment. Hard hat 10 includes a rigid protective layer, shown as outer shell 12. Outer surface 14 of outer shell 12 faces away from the head of the person wearing hard hat 10, and inner surface 16 of outer shell 12 faces inwards towards cavity 20 of hard hat 10. Inner surface 16 defines cavity 20 configured to receive the head of the person wearing hard hat 10. When worn, hard hat 10 is placed on a head of the wearer so that the head is at least partially contained within cavity 20. A compressible inner layer, shown as padding 18, is coupled to inner surface 16 of outer shell 12. In a specific embodiment, padding 18 comprises foam. In various embodiments, a hard hat system 8 includes hard hat 10 and respirator 50, described in more detail below, which are used contemporaneously and/or in conjunction with each other.

The hard hat 10 includes monitoring system that includes a monitoring unit, such as a controller, shown as microcontroller unit (MCU) 22, coupled to hard hat 10. MCU 22 is communicatively coupled to one or more sensors and/or measuring devices, shown as measuring devices 25, via communication links, shown as wire(s) 24. In other embodiments, MCU 22 and one or more of the measuring devices 25 communicate via a variety of communication links, including wireless communication links (e.g., near-field communications (NFC), Bluetooth™). In various embodiments the measuring devices 25 are coupled to the hard hat 10 at locations 30 configured to couple with one or more measuring devices 25. In various embodiments, hard hat 10 includes a plurality of locations 30 configured to couple with one or more measuring devices 25, and a biometric measuring device 26 is coupled to a first location of the plurality of locations 30 and an environmental measuring device 27 is coupled to a second location of the plurality of locations 30. MCU 22 is enabled to communicate with other devices, such as respirator 50 and measuring devices coupled to respirator (described below), and/or other clothing with monitoring systems (e.g., boots) via wired or wireless communications. In a specific embodiment, MCU 22 is coupled to a back or rear portion of hard hat 10, such that MCU 22 is supported adjacent the back of a user's head when hard hat 10 is worn. In various embodiments MCU 22 is configured to analyze one or more biometric characteristics and/or one or more atmospheric conditions, and MCU 22 is further configured to generate an alarm as a result of analyzing the biometric characteristic(s) and/or the atmospheric condition(s).

In various embodiments, the one or more measuring devices 25, supported by outer shell 12 of hard hat 10, include one or more biometric measuring devices 26 that are configured to monitor biometric characteristics of a wearer (e.g., a person) wearing hard hat 10. For example, the one or more biometric measuring devices measure one or more of (e.g., a biometric characteristic selected from the group consisting of) a temperature of the wearer (e.g., of the skin of the wearer near the biometric monitoring device), a heart rate of the wearer, an oxygen saturation (SpO2) of the wearer, and/or a galvanic skin response measurement of the wearer.

In a specific embodiment, the one or more measuring devices 25 include one or more environmental measuring devices 27 configured to measure the environment, such as one or more atmospheric conditions, and generate a signal indicating the results of the measurement. For example, the one or more environmental measuring devices 27 measure a temperature of ambient air around the wearer (e.g., external to outer surface 14 of hard hat 10), and/or a humidity of ambient air around the wearer, etc. Stated another way, in various embodiments the atmospheric condition is selected from the group consisting of a temperature of ambient air around the hard hat and a humidity of ambient air around the hard hat.

In a specific embodiment, the one or more measuring devices 25 include one or more acceleration measuring devices, shown as accelerometer 29, that monitor the environment. For example, the accelerometer 29 monitor an acceleration of the hard hat 10 and generates a signal to MCU 22 indicating a measurement of the acceleration. In a specific embodiment, the accelerometer 29 includes a digital accelerometer to detect sudden impacts to the hard hat 10 and/or the wearer. In various embodiments the MCU 22 is configured to generate an alarm as a result of analyzing the measurement of the acceleration.

In various embodiments, hard hat 10 includes one or more monitoring devices that are selected from biometric measuring devices, environmental measuring devices and/or positional measuring devices. In various embodiments, hard hat 10 includes multiple locations configured to receive a monitoring device that is physically coupled to hard hat 10 and communicatively coupled to MCU 22. For example, a user may have a hard hat 10 that only monitors biometric data and the user would like to also monitor environmental data. In this situation, the user can obtain and couple an environmental monitoring device to hard hat 10, at which point hard hat 10 will also begin monitoring the environmental data received from the environmental monitoring device.

It should be understood that the wearer can add any of the previously mentioned measuring devices to hard hat 10 to increase the monitoring capabilities of hard hat 10. It should also be understood that the wearer can remove a measuring device from hard hat 10 to reduce and/or eliminate a portion of the data being monitored by hard hat 10.

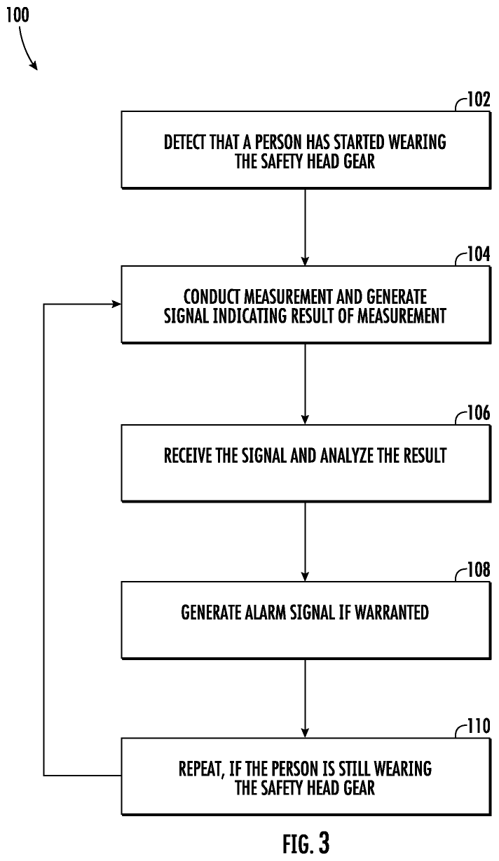

Referring to FIG. 3, depicted is an exemplary method 100 of operating an electronic monitoring system of hard hat 10, according to an exemplary embodiment. Starting at step 102, hard hat 10 detects that a person has started wearing safety headwear, such as hard hat 10 and/or respirator 50 (described below). For example, MCU 22 of hard hat 10 may include a proximity detector that determines when a head of wearer is inside cavity 20. As another example, MCU 22 of hard hat 10 may determine when a heart rate and/or a temperature of wearer can be determined.

At step 104, a measuring device 25 is coupled to hard hat 10 obtains a measurement and generates a signal to MCU 22 that indicates the result of the measurement. For example, a biometric measuring device 26 may begin measuring the temperature of the wearer and send a signal to the MCU 22 that indicates the result of the temperature measurement (e.g., 99.0 degrees F.). In various embodiments, one or more measuring devices 25 are coupled to hard hat 10 and generate a corresponding one or more signals that indicate results of the measurements.

At step 106, MCU 22 receives the one or more signals and analyzes the result(s) of the measurement(s). At step 108, MCU 22 generates an alarm signal as a result of one or more conditions occurring. In a specific embodiment, the alarm signal generates a sound alert to the wearer that an alarm condition has occurred (e.g., a prerecorded message that identifies the error condition).

For example, if MCU 22 determines that a heart rate of wearer is above a predetermined threshold, the MCU 22 generates an alarm signal to communicate to the user that their heart rate is too high and they should consider resting. As another example, if MCU 22 determines that the SpO2 of the wearer is below a predetermined threshold, the wearer should consider resting to catch his/her breath.

As another example, MCU 22 could analyze two different results to generate an error condition. For example, if the wearer of the heart rate is above a predetermined threshold for a predetermined amount of time, and the humidity of the ambient air around hard hat 10 is above a predetermined threshold, the MCU 22 generates an alarm signal to warn the wearer to rest.

At step 110, the monitoring process (e.g., steps 104, 106 and 108) is continued if it is determined that the hard hat 10 is still being worn by the wearer.

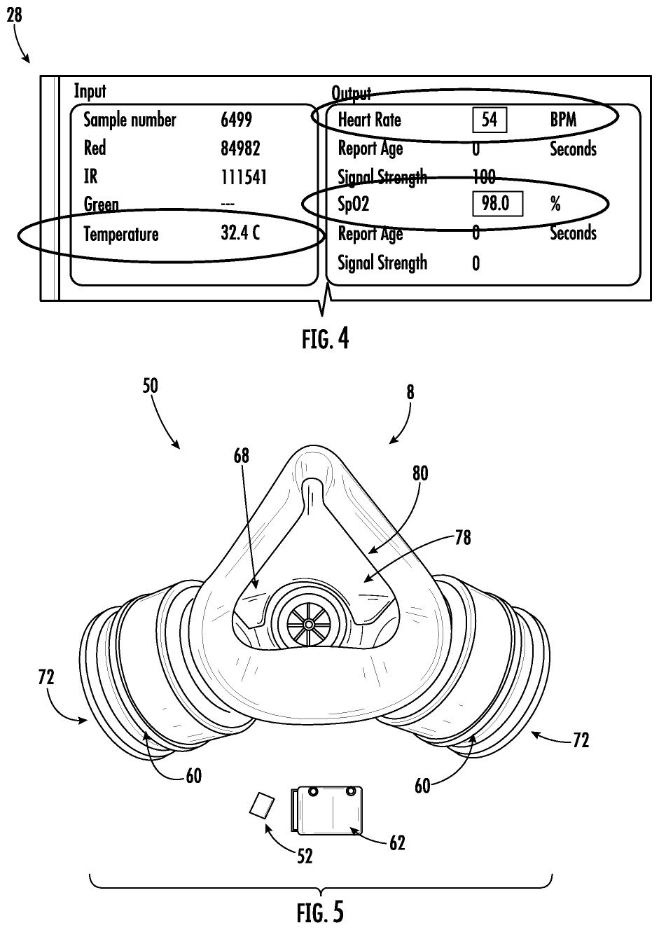

Referring to FIG. 4, various aspects of data being monitored by hard hat 10 are depicted. In one example, hard hat 10 monitors at least a temperature of a wearer, a heart rate of the wearer, and the SpO2 of the wearer, and the results 28 of the measurements are stored and/or communicated via one or more electronic signals.

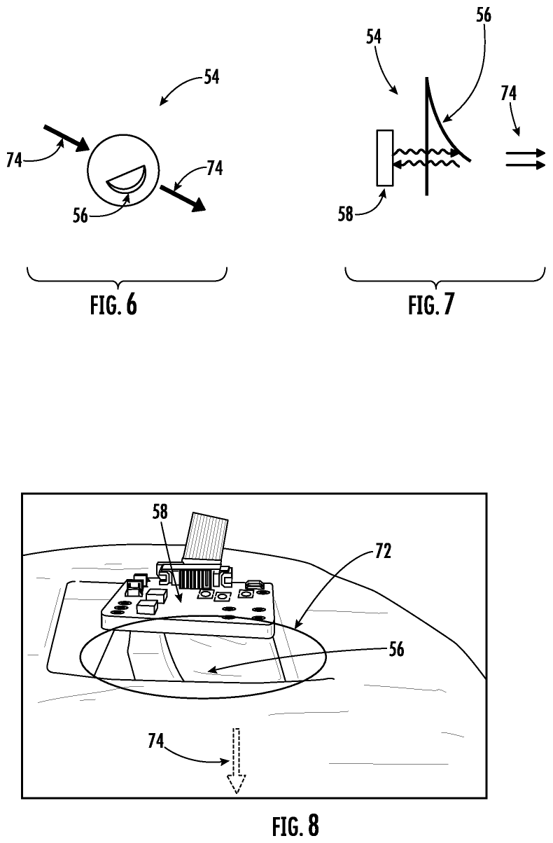

Referring to FIG. 5-8, various aspects of a device for protecting a wearer from particulates in the air, shown as respirator 50, are shown. In a specific embodiment, respirator 50 is used in combination with hard hat 10 such that one or more components of respirator 50 (e.g., a monitoring unit, shown as monitoring unit 62, and/or a measuring device, shown as air pressure measuring device 52) are communicatively coupled with MCU 22 of hard hat 10.

Respirator 50 includes a body 68 and one or more filters 60 configured to remove particulates from air as the air passes through filter 60 to secured area 78. In various embodiments filters 60 are coupled to and supported by body 68 of respirator 50. Secured area 78 is defined between respirator 50 and a face of wearer. When respirator 50 is being worn properly there is an airtight or nearly airtight seal between the face of wearer and gasket 80. Gasket 80 is coupled to body 68 and configured to engage against a face of a person wearing the respirator 50 to define, at least in part, secured area 78 between the respirator 50 and the face of the person. In various embodiments, respirator 50 is coupled to hard hat 10 and monitoring unit 62 of respirator 50 is in communication with MCU 22 of hard hat 10.

When the wearer inhales, negative pressure is temporarily created between the wearer and respirator 50 (e.g., in secured area 78). This negative pressure pulls air in the one or more inlets 72 and through the one or more filters 60, thereby providing fresh air within secured area 78 for the wearer to breath.

When the seal between respirator 50 and the face of wearer has an opening (e.g., if a portion of gasket 80 is not touching the skin of wearer), then the negative pressure within secured area 78 will be refilled by both filtered air transiting filters 60 and unfiltered air transiting the opening. As a result of unfiltered air transiting the opening, the negative pressure within secured area 78 will be normalized (e.g., brought equal to normal atmospheric pressure) quicker than if the seal of respirator 50 is tight and all and/or nearly air entering secured area 78 passes through filters 60.

To detect this situation, respirator 50 includes one or more measuring devices to perform measurements and generate one or more signals based on those measurements. Air pressure measuring device 52 monitors an air pressure between respirator 50 and a face of wearer, such as the air pressure within secured area 78. In various embodiments, central processing unit 62 and/or by MCU 22 compares the air pressure measurement to a predetermined value and generates an alarm in response to the comparison determining the air pressure measurement is lower than the predetermined value. In a specific embodiment, air pressure measuring device 52 generates one or more signals that indicate the air pressure measurements obtained by air pressure measuring device(s) 52. The one or more signals are received by central processing unit 62 and/or by MCU 22 (e.g., via step 106 in FIG. 3).

Central processing unit 62 and/or by MCU 22 receive and analyze the one or more signals to determine if the seal between respirator 50 and the face of wearer is sufficiently airtight. Central processing unit 62 is supported by body 68. In various embodiments central processing unit 62 and/or by MCU 22 is configured to receive a signal from air pressure measuring device 52 indicating the air pressure measurement, and further configured to generate an alarm in response to detecting an error condition based on analyzing the air pressure measurement. In a specific embodiment, a pressure measurement is performed before respirator 50 is applied to a face of user to determine normal atmospheric pressure (e.g., as a calibration measurement to be compared against future measurements). As a result of determining the seal between respirator 50 and the face of wearer is not sufficiently airtight, the central processing unit 62 generates a signal that generates an alarm (e.g., a prerecorded message that respirator 50 should be adjusted to improve the seal).

A measurement device, shown as air volume measuring device 54, is coupled to respirator 50 and in fluid communication with one or more of filters 60. In a specific embodiment, air volume measuring device 54 is configured to measure a volume of air, such as a volume of air transiting filter 60, and generate a signal (e.g., an electronic signal) indicating the measured volume. In various embodiments, air volume measuring device 54 includes a device for monitoring a volume of air, such as a projection configured to deflect, shown as flap 56. In response to air transiting air volume measuring device 54 in direction 74, the air deflects flap 56. A detector, shown as photodetector 58, monitors an amount that flap 56 is deflected. The more that flap 56 is deflected, the more air is estimated to be transiting past flap 56.

An estimate of the volume of air transiting flap 56 can be calculated by measuring the deflection of flap 56. For example, air flow can be determined by multiplying air velocity times surface area of flap 56 (e.g., air flow Q=velocity*surface area).

In a specific embodiment, filter 60 is in fluid communication with and in series with air volume measuring device 54, so the amount of air transiting air volume measuring device 54 is equal to the amount of air transiting filter 60. The performance characteristics of filter 60 can be estimated by monitoring the volume of air transiting filter 60 and the air pressure of respirator 50 (e.g., the air pressure of secured area 78). As filter 60 becomes more clogged with particulates and other objects interfering with air transiting filter 60, less air will transit through filter 60 (and therefore also flap 56) for a given negative air pressure in respirator 50. The resistance of filter 60 can be determined by dividing the air pressure measurement by the air flow Q (e.g., Resistance=Pressure/air flow Q).

In a specific embodiment, one or more components in respirator 50 are communicatively coupled to hard hat 10. For example, air pressure measuring device 52 of respirator 50 is communicatively coupled with MCU 22 of hard hat 10, and MCU 22 of hard hat 10 analyzes signals received from air pressure measuring device 52.

The monitoring unit 62 monitors the air pressure and the air volume transiting filter 60. When the air volume is below a threshold (e.g., when filter 60 is too clogged and/or dirty), monitoring unit 62 will generate an alarm signal to the user to replace the filter). In a specific example of this process, the MCU 22 and/or the monitoring unit 62 may receive a signal indicating a measured volume (of air transiting filter) and a third signal that identifies the filter, the MCU 22 and/or the monitoring unit 62 analyzes the signal and is further configured to generate an alarm to replace the filter as a result of analyzing the second signal and the third signal. In a specific embodiment, the signal indicating a measure volume is based on a velocity of air transiting the air volume measuring device 54, and a surface area of a flap 56 that deflects in response to air through the filter 60, and the air volume measuring device 54 measures an amount of deflection of the flap 56 and analyzes the measured deflection to calculate an estimated velocity of the air transiting the filter 60.

In another example, filter 60 includes a component that uniquely identifies the filter, for example an electrically erasable programmable read-only memory (EEPROM) chip, such as via a serial number. In various embodiments, the MCU 22 and/or the monitoring unit 62 is configured to receive a signal that uniquely identifies filter 60. When filter 60 is coupled to respirator, monitoring unit 62 reads the serial number of filter 60. As a filter 60 is being used, monitoring unit 62 measures and/or calculates a total amount of time that filter 60 is in use. When a predetermined threshold is reached (e.g., after 100 hours), an alarm signal is generated to alert the user to replace filter 60 as a result of the calculated amount of time exceeding a predetermined threshold. In various embodiments, the alarm signal provided to the user, is one or more of an auditory signal, a vibratory signal, and/or a visual signal.

In various embodiments, the MCU 22 and/or the monitoring unit 62 is configured to generate an alarm in response to analyzing the combination of a signal indicating the measurement of the biometric characteristic and a third signal indicating the measurement of an atmospheric condition. For example, signal indicating the biometric characteristic may be a measurement of a temperature of the person wearing the hard hat, and the signal indicating a measurement of the atmospheric condition may be a temperature of ambient air around the hard hat.

Referring to FIG. 9, depicted are exemplary measurements of air pressure readings of respirator 50, according to an embodiment. Air pressure within respirator 50 is periodically measured and indicated via air pressure measurements 64. Air pressure measurements 64 is supported by body 68 and configured to analyze an air pressure measurement within the secured area 78 to determine if an error condition is and/or can be detected based on an analysis of the measurement(s). As the wearer inhales, the pressure dips lower until air reenters secured area 78, such as via filter 60. As the wearer exhales, the pressure raises until air exits secured area 78, such as via an outlet. A modified measurement, such as a moving average 66 of air pressure measurements 64 can be compared to a predetermined threshold 70. As a result of moving average 66 exceeding the predetermined threshold 70, an alarm signal is generated to alert the wearer that respirator 50 may need to be adjusted to improve the seal between respirator 50 and the face of the wearer. In various embodiments, the MCU 22 and/or the monitoring unit 62 is configured to receive a signal indicating the moving average 66 of the air pressure measurement and to generate an alarm in response to detecting an error condition via analyzing the moving average of the air pressure measurement.

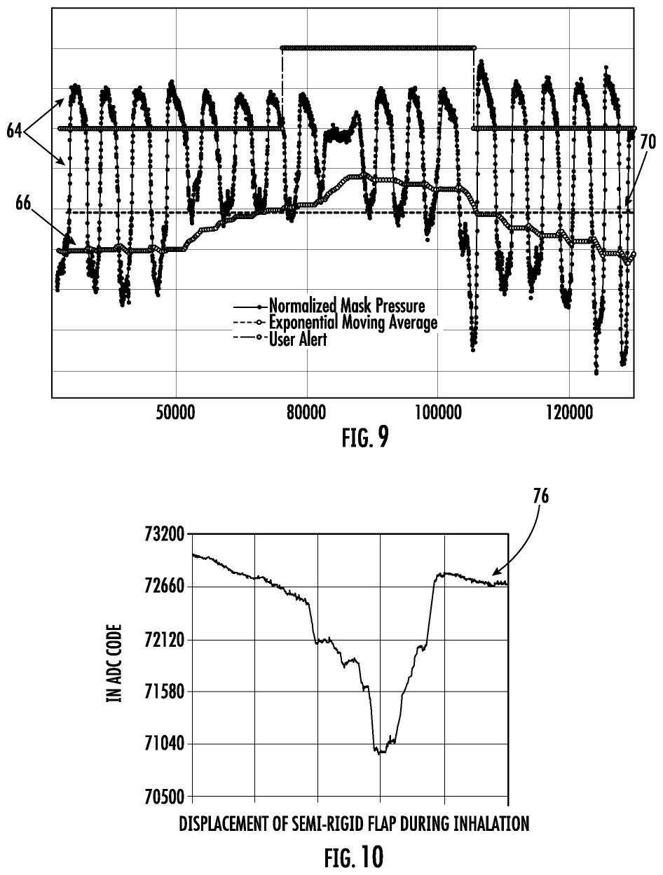

Referring to FIG. 10, depicted are exemplary displacement measurements of flap 56, according to an embodiment. As the wearer inhales, air enters secured area 78 past flap 56, which displaces the flap 56 (the center of the graph) a distance until air pressure in secured area 78 is normalized with the air pressure outside of respirator 50 (the right side of the graph).

It should be understood that the figures illustrate the exemplary embodiments in detail, and it should be understood that the present application is not limited to the details or methodology set forth in the description or illustrated in the figures. It should also be understood that the terminology is for description purposes only and should not be regarded as limiting.

Further modifications and alternative embodiments of various aspects of the disclosure will be apparent to those skilled in the art in view of this description. Accordingly, this description is to be construed as illustrative only. The construction and arrangements, shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present disclosure.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred. In addition, as used herein, the article “a” is intended to include one or more component or element, and is not intended to be construed as meaning only one. As used herein, “rigidly coupled” refers to two components being coupled in a manner such that the components move together in a fixed positional relationship when acted upon by a force.

Various embodiments of the disclosure relate to any combination of any of the features, and any such combination of features may be claimed in this or future applications. Any of the features, elements or components of any of the exemplary embodiments discussed above may be utilized alone or in combination with any of the features, elements or components of any of the other embodiments discussed above.

For purposes of this disclosure, the term “coupled” means the joining of two components directly or indirectly to one another. Such joining may be stationary in nature or movable in nature. Such joining may be achieved with the two members and any additional intermediate members being integrally formed as a single unitary body with one another or with the two members or the two members and any additional member being attached to one another. Such joining may be permanent in nature or alternatively may be removable or releasable in nature.

While the current application recites particular combinations of features in the claims appended hereto, various embodiments of the invention relate to any combination of any of the features described herein whether or not such combination is currently claimed, and any such combination of features may be claimed in this or future applications. Any of the features, elements, or components of any of the exemplary embodiments discussed above may be used alone or in combination with any of the features, elements, or components of any of the other embodiments discussed above.

What is claimed is:

1. A hard hat comprising:
an outer shell;
an outer surface of the outer shell;
an inner surface of the outer shell, the inner surface defining a cavity configured to receive a head of a person wearing the hard hat;
a biometric measuring device supported by the outer shell and configured to measure a biometric characteristic of the person wearing the hard hat; and
an environmental measuring device supported by the outer shell and configured to measure an atmospheric condition.

2. The hard hat of claim 1, comprising a monitoring unit configured to analyze the biometric characteristic and/or the atmospheric condition, wherein the monitoring unit is configured to generate an alarm as a result of analyzing the biometric characteristic and/or the atmospheric condition.

3. The hard hat of claim 2, wherein the atmospheric condition is selected from the group consisting of a temperature of ambient air around the hard hat and a humidity of ambient air around the hard hat.

4. The hard hat of claim 2, further comprising an acceleration measuring device that measures an acceleration of the hard hat and generates a signal to the monitoring unit indicating a measurement of the acceleration, the monitoring unit is configured to generate an alarm as a result of analyzing the measurement of the acceleration.

5. The hard hat of claim 1, wherein the measurement of the biometric characteristic comprises a galvanic skin response measurement.

6. The hard hat of claim 1, wherein the biometric characteristic is selected from the group consisting of a heart rate of the person wearing the hard hat, a temperature of the person wearing the hard hat, and an oxygen saturation (SpO2) of the person wearing the hard hat.

7. The hard hat of claim 1, the outer shell comprising a plurality of locations each configured to couple with a measuring device, wherein the biometric measuring device is coupled to a first location of the plurality of locations, and wherein the environmental measuring device is coupled to a second location of the plurality of locations.

8. A respirator comprising:
a body;
a gasket coupled to the body and configured to engage against a face of a person wearing the respirator to define a secured area between the respirator and the face of the person;
a filter coupled to the body and configured to remove particulates from air passing through the filter to the secured area;
an air pressure measuring device supported by the body and configured to measure an air pressure measurement within the secured area and generate a signal indicating the measured air pressure measurement; and
a monitoring unit supported by the body and configured to receive the signal from the air pressure measuring device indicating the air pressure measurement, the monitoring unit configured to generate an alarm in response to detecting an error condition based on analyzing the air pressure measurement.

9. The respirator of claim 8, wherein the monitoring unit analyzes the signal via comparing the air pressure measurement to a predetermined value, and the monitoring unit generates the alarm in response to the comparison determining the air pressure measurement is lower than the predetermined value.

10. The respirator of claim 9, comprising an air volume measuring device configured to measure a volume of air transiting the filter and generate a second signal indicating the measured volume.

11. The respirator of claim 10, the monitoring unit further configured to receive the second signal indicating the measured volume and a third signal that identifies the filter, the monitoring unit further configured to generate an alarm to replace the filter as a result of analyzing the second signal and the third signal.

12. The respirator of claim 10, wherein the second signal is based at least in part on:
a velocity of air transiting the air volume measuring device; and
a surface area of a projection that deflects in response to air through the filter, wherein the air volume measuring device configured to measure an amount of deflection of the projection and configured to analyze the measured deflection to calculate an estimated velocity of the air transiting the filter.

13. The respirator of claim 8, the monitoring unit configured to:
receive a signal that uniquely identifies the filter;
calculate an amount of time the filter has been in use; and
generate an alarm signal as a result of the calculated amount of time exceeding a predetermined threshold.

14. The respirator of claim 10, the monitoring unit is configured to receive a signal indicating a moving average of the air pressure measurement and to generate an alarm in response to detecting an error condition via analyzing the moving average of the air pressure measurement.

15. A hard hat system comprising:
a respirator comprising:
a body;
a gasket coupled to the body and configured to engage against a face of a person wearing the respirator to define a secured area between the respirator and the face of the person;
a filter coupled to the body and configured to remove particulates from air passing through the filter to the secured area; and
an air pressure measuring device supported by the body and configured to generate a first signal indicating an air pressure measurement; and
a hard hat comprising:
an outer shell;
an inner surface of the outer shell, the inner surface defining a cavity configured to receive a head of the person wearing the hard hat and the respirator;
a biometric measuring device supported by the outer shell, the biometric measuring device configured to generate a second signal indicating a measurement of a biometric characteristic of the person wearing the hard hat; and
a monitoring unit supported by the outer shell and communicably coupled to both the air pressure measuring device and the biometric measuring device, the monitoring unit configured to receive and analyze the first signal and the second signal, the monitoring unit configured to generate a first alarm in response to analyzing one or more of the first signal and the second signal.

16. The hard hat system of claim 15, the hard hat comprising an environmental measuring device supported by the outer shell and configured to generate a third signal indicating a measurement of an atmospheric condition.

17. The hard hat system of claim 16, the monitoring unit configured to generate a second alarm in response to analyzing the combination of the second signal indicating the measurement of the biometric characteristic and the third signal indicating the measurement of an atmospheric condition.

18. The hard hat system of claim 17, the second signal indicating a measurement of a temperature of the person wearing the hard hat, and the third signal indicating a measurement of a temperature of ambient air around the hard hat.

19. The hard hat system of claim 15, the monitoring unit is configured to receive a signal indicating a moving average of the air pressure measurement and to generate a third alarm in response to detecting an error condition via analyzing the moving average of the air pressure measurement.

20. The hard hat system of claim 15, wherein the measurement of the biometric characteristic comprises a measurement of an oxygen saturation (SpO2) of the person wearing the hard hat.
